(19) United States
(12) Patent Application Publication
(10) Pub. No.: US 2010/0004321 A1
(43) Pub. Date: Jan. 7, 2010

(54) Recombinant Staphylococcal Phage Lysin as an Antibacterial Agent

(76) Inventors: Paul Ross, County Cork (IE); Aidan Coffey, County Cork (IE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/308,796

(22) PCT Filed: Jun. 29, 2007
(86) PCT No.: PCT/IE2007/000064

§ 371 Date: Jun. 18, 2009

(30) Foreign Application Priority Data
Jun. 29, 2006 (IE) 2006/0488

(51) Int. Cl. A61K 31/7088; C12N 15/74; C07H 21/04; C07K 14/00; C12N 1/06; C12N 1/00; A61P 31/04

(52) U.S. Cl. 514/44 R; 435/320.1; 536/23.1; 530/350; 435/259; 435/243

(57) ABSTRACT

The present invention provides a plasmid pSOFLysK contained in the bacterial strain Lactococcus lactis NZ9800 referred herein as Lactococcus lactis NZ9800-pSOFLysK (subsequently designated Lactococcus lactis DPC6132) encoding anti-staphylococcal activity as deposited with DSMZ under accession No. ncimb 41409 and plasmids substantially similar thereto also providing anti-staphylococcal activity. In another aspect, the present invention provides a gene encoding an anti-staphylococcal protein, Lysin (LysK) as encoded by the plasmid pSOFLysK in Lactococcus lactis/VZ9800-pSOFLysK (subsequently designated Lactococcus lactis DPC6132). The recombinant lysine also has applications in diagnostics given its lytic mechanism.

### FIELD OF THE INVENTION

The present invention relates to the field of cloning of recombinant lysin from a staphylococcal bacteriophage. In particular, the present invention relates to the use of recombinant staphylococcal lysin (LysK) cloned from staphylococcal bacteriophage K and fractions thereof as an antimicrobial agent for killing a wide range of staphylococci in addition to using it for diagnostic applications.

### BACKGROUND TO THE INVENTION

The increasing prevalence of antibiotic resistance in clinical isolates of Staphylococcus aureus is a major problem, given that the bacterium causes a wide variety of human infections ranging from simple abscesses to fatal sepsis, as well as endocarditis, pneumonia, mastitis, phlebitis, meningitis and toxinosis (for review see 24), in addition to a wide range of animal diseases.

The rapid emergence of penicillin resistant S. aureus in the 1950s lead to the use of methicillin and related drugs for treatment of infections. In the 1960s, methicillin-resistant S. aureus (MRSA) emerged and have since become endemic in many hospital environments (14). In addition, these MRSA strains also frequently exhibit resistance to a variety of other common antibiotics (20).

Indeed, over 95% of patients worldwide with S. aureus infections do not respond to first-line antibiotics, for example ampicillin and penicillin (33). Recently, the SENTRY Antimicrobial Surveillance Program reported that 36.8% of S. aureus isolates ribotyped belonged to the multidrug-resistant, oxacillin-resistant S. aureus species (7).

In Ireland, Naylor et al (23) found that MRSA was the commonest single organism cultured from patients with complex wound and graft infections after vascular surgery. In addition, the latest data from the European Antimicrobial Resistance Surveillance System showed an increase in MRSA from 39% in 1999 to 45% in 2002 in Ireland (37).

Until recently, S. aureus has exhibited sensitivity to the glycopeptide antibiotics vancomycin and teicoplanin and therefore these antibiotics represent one of the last lines of defence available against staphylococcal infection. However, the recent emergence of vancomycin-resistant S. aureus (VRSA) and also teicoplanin resistant strains in hospital infections poses a major threat to this approach (13). As a result, investigations for new and alternative anti-microbials effective against S. aureus have become increasingly relevant.

Bacteriophages (phage) were investigated as far back as 1921 to eliminate bacteria including staphylococci in human infections (35). The majority of documented human phage therapy studies have been performed in Poland (29) and the former Soviet Union and these include challenges against Staphylococcus aureus (for review see 36). In the case of S. aureus the potential of phage as an antibacterial therapeutic was clearly shown by Matsuzaki and co-workers (21), who significantly reduced mortality numbers of mice previously injected with S. aureus by intraperitoneal injections of phage MR11. Moreover, since the early 1990s, a variety of new companies have been established worldwide that have placed major emphasis on bacteriophage research with the aim of treating multi-drug resistance bacteria causing infections. Phage K is a polyvalent phage with a broad host range, inhibiting both coagulase positive and negative staphylococci (32). It is a member of the family Myoviridae (1) and has been the subject of previous studies (15-17, 28-30). The origin of phage K is unclear. Both Rountree in 1949 (32) and Rippon in 1956 (31) state that phage K of Krueger (18) is identical to phage Au2 described by Burnet and Lush in 1935 (4), Burnet and Lush also state that the phage used by Krueger in 1930-31 (18) is Au2 and suggest that phage Au2 could be derived from Gratia's H strain of S. aureus from 1922 (11), but noted that this derivation is not positively known (4).

Although research on phage therapy diminished outside of the former Soviet Union with the advent of antibiotics, it has been revisited primarily as a result of the antibiotic resistance problem. This renewed interest is evident from the number of reviews published recently (2, 3, 5, 8, 9, 19, 22, 26 and 36).

Recently, lytic enzymes associated with the phage and known phage lysins have attracted considerable interest as novel anti-microbials against gram-positive bacteria. These phage encoded enzymes allow the phage to escape from an infected bacterial cell by degrading the bacterial cell wall. Where such enzymes have been purified, they have been demonstrated to effectively kill a range of pathogenic bacteria such as group A streptococci (39) Streptococcus pneumonia (40), Bacillus anthracis (41) and Enterococcus faecalis (51). A staphylococcal lytic enzyme called virolysin was previously identified in phage lysates but this only showed activity against dead and not live cells (43). Another account of lysin activity, associated with culture media after phage lysis, was reported by Sonstein et al (42) and designated PAL (phage associated lysin). While this enzyme activity worked against live S. aureus cells and was characterised as having peptidase activity, no therapeutic or biocontrol capabilities were suggested (42). In addition, phage lytic enzymes from staphylococcal phages Twort (44, 45), phi11 (46) and 80α (47) have previously been described but neither their ability to kill live cells nor their possible therapeutic capabilities have been reported.

### OBJECT OF THE INVENTION

It is an object of the present invention to produce recombinant staphylococcal phage lysin known as LysK. This invention also concerns the method of cloning, characterisation and expression of the lysin (LysK) from staphylococcal phage K into Lactococcus lactis NZ9800. The resulting strain has been designated Lactococcus lactis DPC6132 and is essentially Lactococcus lactis NZ9800 containing the recombinant plasmid pSOFLysK. It is a further object of the invention to evaluate the efficacy of recombinantly produced LysK in the elimination of pathogenic staphylococcal bacteria including a number of coagulase positive and negative staphylococci associated with bovine infections and also antibiotic resistant S. aureus associated with human infections including MRSA and VRSA. This invention thus provides new and alternative antimicrobials that are effective against pathogenic staphylococci. In addition, the invention provides a convenient approach to lysing staphylococci for diagnostic applications.

### SUMMARY OF THE INVENTION

The present invention provides a plasmid pSOFLysK contained in the bacterial strain Lactococcus lactis NZ9800 referred herein as Lactococcus lactis NZ9800-pSOFLysK (subsequently designated Lactococcus lactis DPC6132) encoding anti-staphylococcal activity as deposited with NCIMB under accession no NCIMB 41409 on 8 Jun. 2006 and plasmids substantially similar thereto also providing anti-staphylococcal activity. In another aspect, the present invention provides a gene encoding an anti-staphylococcal protein, Lysin (LysK) as encoded by the plasmid pSOFLysK in Lactococcus lactis DPCNZ9800 and designated Lactococcus lactis DPC6132.

The plasmid described in the present invention may be extremely useful for cloning large (amplified) quantities of genetic material providing anti-staphylococcal activity. Preferably the plasmid may be an expression vector replicating in Escherichia coli or Lactococcus lactis or another bacterial genus. Desirably plasmids amplifying the genetic material encoding anti staphylococcal activity are under the control of a promoter signal for example the T7 promoter or the nisin (nisA) promoter or the like. Preferably, the genetic material providing anti-staphylococcal activity may be derived from the genome of phage K or another similar staphylococcal phage. It will be apparent to a person skilled in the art that use of the term genetic material includes RNA such as mRNA, rRNA tRNA or DNA such as cDNA, plasmid DNA, mitochondrial DNA genomic DNA and the like.

The present invention also:

relates to use of a plasmid encoding anti-staphylococcal lysin activity as contained in the bacterial strain Lactococcus lactis NZ9800 and designated Lactococcus lactis DPC6132 as deposited with the DSMZ under accession No. NCIMB 41409 on 8 Jun. 2006 and plasmids substantially similar thereto also encoding anti-staphylococcal activity; or
a gene encoding an anti-staphylococcal protein as contained in plasmid pSOFLysK in Lactococcus lactis DPCNZ9800, designated Lactococcus lactis DPC6132 as deposited with the NCIMB under accession No. NCIMB 41409 and genes substantially similar thereto also encoding anti-staphylococcal activity.

The invention also provides a lysin protein encoded by the deposited plasmid pSOFLysK and the N-terminal 161 amino-terminal CHAP domain of that protein. The CHAP domain may have the sequence;

GAIDADGYYHAQCQDLITDYVLWLTDNKVRTWGNAKDQIKQSYGTGFKIH
ENKPSTVPKKGWIAVFTSGSYEQWGHIGIVYDGGNTSTFTILEQNWNG
YA,

or a sequence substantially similar thereto also having lytic activity.

The CHAP domain may be used to produce chimeric proteins in which the CHAP domain is linked with other peptides or proteins to produce a molecule which has lytic activity and additional substrate specificities. Due to the modular design of phage lysins, it is possible to construct “hybrid proteins” by combining different domains from different proteins. These would have different specificities to the original protein. For example, by using varying cell binding domains, the protein could be designed to lyse a range of different bacteria. Modular assembly of functional domains is a rational approach for constructing enzymes with novel properties.

Thus in a still further aspect, the invention provides a chimeric protein comprising the CHAP domain of LysK or a peptide substantially similar thereto also encoding anti-bacterial activity, or a nucleotide sequence encoding such a chimeric protein.

The ultimate application of the protein or CHAP domain may be an injectable-grade pharmaceutical composition, a disinfectant composition or a topical composition such as a topical preparation selected from the group comprising a hand wash, a skin wash, a shampoo, a topical cream, a disinfecting preparation, a bismuth-based cream or the like. The composition may also be used to disinfect an environment. Pharmaceutical compositions may be formulated with pharmaceutically acceptable carriers and diluents.

Desirably, the staphylococci which are targeted by this invention may be selected from the group comprising: Staphylococcus aureus, Staphylococcus epidermidis, Staphylococcus saprophyticus, Staphylococcus chromogenes, Staphylococcus captis, Staphylococcus hominis, Staphylococcus haemolyticus, Staphylococcus caprea, Staphylococcus hyicus and antibiotic-resistant strains (including methicillin and vancomycin resistant staphylococci) and combinations thereof.

In addition the present invention provides a convenient tool to efficiently lyse staphylococci and thus may be very useful for a range of diagnostic applications. By “substantially similar” is meant sequences or molecules which because of degeneracy of the genetic code, or because of other mutations, encode a nucleotide or protein which has the same or similar properties to the molecules defined herein and in particular the anti-bacterial properties or capabilities of such sequences or molecules. In particular, substantially similar molecules have at least about 80% sequence homology under high stringency conditions. The molecules may have at least about 90% homology or at least about 95% homology under high stringency conditions.

### BRIEF DESCRIPTION OF THE DRAWINGS

The invention will be more clearly understood from the following description of some embodiments thereof, given by way of example only, with reference to the accompanying drawings, in which:

FIG. 1. Electron micrograph images of phage K, from which LysK was derived, negatively stained with 1% uranyl acetate. A: Image on left indicates contractile tail. B: Image on right indicates phage K with tail contracted and black phage head. Scale bar represents 100 nm.

FIG. 2A. A zymogram which contains autoclaved MRSA (DPC5645) cells. Lane 1, pre-stained low range molecular weight marker (Bio-rad); lane 2, NZ9800-pNZ8048 without nisin; lane 3, NZ9800-pNZ8048 with nisin; lane 4, NZ9800-pSOFLysK without nisin; lane 5, NZ9800-pSOFLysK with nisin. LysK activity is indicated by a black arrow.

FIG. 2B. Killing of S. aureus DPC5645 with lactococcal lysates containing LysK. Lysates obtained from NZ9800-pSOFLysK with nisin was used as the source for LysK and lysates obtained from NZ9800-pNZ8048 with nisin was used a control. Symbols represent the following: ▪ cell numbers of DPC5645+lysate from induced NZ9800-pNZ8048, ␣ cell numbers of DPC5645+lysate from induced NZ9800-pNZ8048 and ∘ OD values of DPC5645+lysate from induced

NZ9800-pSOFLysK. Values are the means from three independent experiments with standard deviation indicated by vertical bars.

FIG. 3. Schematic representation of phage K lysin and some deletion derivatives. The domains remaining in each of the constructs is depicted; also, the lytic activity associated with each construct is indicated.

### DETAILED DESCRIPTION OF THE DRAWINGS

The lysin, LysK, identified from the genome of phage K, in L. lactis has been cloned and heterologously over-expressed. Phage K (American Type Culture Collection, 19685-B1) is a polyvalent broad-host-range anti-staphylococcal phage. Its genome has been previously sequenced (25, incorporated herein by reference only) and it has been shown to kill a broad range of newly isolated pathogenic staphylococci, including both human and veterinary strains (48, incorporated herein by reference only). Initially LysK was cloned and heterologously over-expressed in Escherichia coli (as a His-tagged fusion protein under the control of the T7 promoter), however, recombinant LysK was consistently located in the insoluble fraction as inclusion bodies (data not shown). For this reason we chose to express the lysin in the gram-positive organism L. lactis NZ9800 (34) using the nisin inducible expression (NICE) system (49, incorporated herein by reference only). In addition to lysing dead staphylococci, a lactococcal lysate containing recombinant LysK inhibited live cultures of a number of pathogenic strains demonstrating the lytic capabilities of this lysin in controlling staphylococcal numbers.

### Materials and Methods

### Bacterial Strains and Growth Media

Phage K was purchased from the American Type Culture Collection (ATCC 19685-B1). Staphylococcal strains used to assess the host range of phage K are listed in Table 1. Strains with the prefix DPC are held in the Dairy Products Research Centre culture collection. Mu3, Mu50, ST3550, ST2573 and 8325 were purchased from the Public Health Laboratory Service (PHLS, UK). Human MRSA strains were isolated from hospital staff, outpatients and in patients from Irish Hospitals over a three-year period, and are held at the Cork Institute of Technology (Table 1). Strains were grown at 37° C. in Brain Heart Infusion (BHI) broth (Oxoid, UK). Solid media contained 1.0% (w/v) bacteriological agar (Oxoid, UK). All strains were stocked in BHI containing 40% glycerol and stored at −80° C.

### Phage Propagation.

Phage K was routinely propagated on S. aureus DPC5246 in BHI broth. Concentrated phage K preparations were obtained by CsCl density gradient centrifugation following Polyethylene Glycol (m.w. 8000) precipitation of phage lysates of BHI cultures. Phage propagation protocols were used as described previously (25, incorporated herein by reference only). Phage preparations were dialysed in 10 mM sodium phosphate buffer pH 7 and filter sterilised prior to use (0.45 μm). Propagation of phage K on staphylococci, which exhibited reduced phage sensitivity, was achieved by incubating 100 μl of phage K (approx. 10⁸ plaque forming units (p.f.u.)/ml) with 20 mls of BHI containing a 1% inoculum from an overnight culture of the required host strain. Samples were incubated at 37° C. overnight. Samples were then centrifuged and the supernatant filter-sterilised and phage plaque assays repeated. Modified phages were named according to the propagating strain.

### Electron Microscopy

Phage stocks were prepared from CsCl density gradients to achieve titres in excess of 10⁹ p.f.u./ml. Each sample was stained negatively with 1% uranyl acetate and electron micrographs were taken at various magnifications using JEM EX 1200 electron microscope.

### Phage Plaque Assays

Phage plaque assays and phage sensitivity tests were performed as described previously (27, incorporated herein by reference only). Briefly, 50 μl of the appropriate overnight culture, 20 μl of 1 M CaCl₂ and 1 ml of the appropriate phage dilution was added to 5 ml of BHI overlay (0.7% agar). The contents were mixed and poured onto BHI plates and incubated at 37° C. for 18 hours.

### Phage Host Range and Bacterial Challenge

Phage K was assessed for its ability to form a clearing on a lawn of each of the staphylococcal strains. The lawn was prepared by adding 50 μl of overnight culture (grown from a 1% inoculum with shaking at 37° C.) to a molten 4-ml agar (0.7%) overlay based on BHI medium (Oxoid, U.K.), which was poured over the BHI plate. After the overlay had solidified, a 10 μl aliquot of phage was spotted onto the surface. Plates were dried and incubated at 37° C. for 18 hours. Clearing indicated phage sensitivity. Results were confirmed by the plaque assay technique (above). Phage challenge experiments were performed in BHI broth with shaking at 100 rpm at 37° C. Generally, overnight cultures were pre-grown in BHI and inoculated into BHI such that the initial titre was approximately 10⁶ colony forming units (c.f.u.)/ml. Phage K was added at a multiplicity of infection (m.o.i.) of 1 after the culture had reached approximately 10⁷ c.f.u./ml. Samples were then removed and plated in triplicate at regular intervals (the lower limit of detection was 10 c.f.u./ml). Plates were incubated overnight at 37° C. Plate counts were recorded in triplicate and standard deviations determined. Phage titre changes over the course of the challenge were monitored by plaque assay simultaneously.

Antibiotic Susceptibility Testing.

The methicillin resistance phenotype of the staphylococcal strains was determined by the use of antibiotic susceptibility discs obtained from OXOID (Basingstoke, Hampshire, United Kingdom). BHI plates were overlaid with each staphylococcal strain after overnight growth. Antibiotic discs were dispensed onto each plate and after overnight incubation at 37° C., each plate was scored for antibiotic sensitivity using the Kirby-Bauer plate method (12, incorporated herein by reference only).

### Sequence Analysis, Cloning and Over-Expression of Lysk.

To amplify lysK for cloning and plasmid constructions, cDNA was used as the template as the lysin gene is interrupted by an intron (25, incorporated herein by reference only). RNA was isolated and cDNA synthesised as described previously (25, incorporated herein by reference only). RT-PCR results demonstrated that the lysK transcript appears between 10 and 20 min after phage infection (data not shown). The lysK gene was amplified from phage K cDNA using the following primers: lysinF (5′CGG CAT GCA GGA GGA AAA AAA AM TGG CTA AGA CTC MG CAG AAA TAA ATA AAC 3′) and LysinR (5′ GCTCTA GAC TAT TTG MT ACT CCC CAG GC 3′) and cloned into the SphI/XbaI sites of the nisin expression vector pNZ8048 generating the plasmid pSOFlysK. This construct was introduced into E. coli XL-1 blue and checked for the correct sequence and subsequently introduced into L. lactis NZ9800 an MG1614 derivative containing the nisRK signal transduction genes integrated on the chromosome. When compared with sequences in the database, LysK was found to contain both a domain from the amidase-2 (N-acetylmuramoyl-L-alanine amidase) family and a CHAP (cysteine, histidine-dependent amidohydrolases/peptidases) domain.

### Deletion Analysis of Lysk

To analyze how the structure of LysK relates to its function, a number of deletion derivatives of LysK were constructed. Constructs were designed so as to remove various functional domains from the C-terminal end of the intact protein. PCR with Expand High Fidelity Taq Polymerase (Roche) was used to amplify the desired regions of LysK, according to the manufacturer's recommendations. The oligonucleotide primers used for these PCR reactions are listed in Table 3. Where appropriate, splicing by overlap extension (SOEing) PCR was used in the synthesis of constructs with internal deletions. Site-directed mutagenesis of active site amino acids was performed with the Quikchange XL mutagenesis kit from Stratagene. All inserts were cloned into the pTOPO vector (Invitrogen) and sequenced to confirm their integrity. Inserts were then excised from pTOPO and cloned into the NcoI/BgIII sites of the pQE60 (Qiagen) expression vector. The resulting plasmids were then transformed into E. coli XL1 Blue. SDS-PAGE and zymogram assays were used to visualize the activity of LysK and its deletion derivatives in pQE60 and to assess which domain(s) the lytic activity of LysK could be ascribed to.

### Results

### Phage K Exhibits Morphology of the Myoviridae.

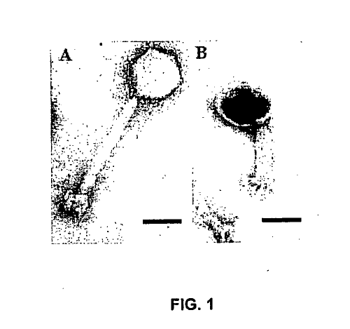

In a previous study we have shown that phage K is the founding member of a new taxonomic group within the Myoviridae family based on molecular characterisation of the similarity between phage genomes (25, incorporated herein by reference only). The morphology of phage K supports this grouping in that electron microscopy exhibits characteristics of the Myoviridae family. Electron micrographs show that phage K has an isometric head with contractile tail (FIG. 1a and 1b). Also, the basal tuft of phage K is evident, FIG. 1b clearly shows knob like appendages extending from the baseplate. In this electron micrograph (FIG. 1b.) the tail is contracted, the DNA has been ejected (head is black) and the protruding core of the tail is evident.

### Phage K Inhibits Recently Emerged Drug Resistant Bacteria

Phage K does not require the addition of CaCl₂ to BHI in order to infect, since there was no difference in plaque forming ability when CaCl₂ was omitted from the plaque assays. In addition increasing the concentration of CaCl₂ (0.1, 0.5, 1, 5, 10 and 20 mM) had no effect on plaque forming ability (Data not shown). To test the host range and potency of phage K bacterial challenge experiments were performed. Details of the bacterial strains are shown in Table 1. These include a S. aureus type strain, 36 human MRSA strains, 4 glycopeptide resistant strains, 4 distinct clinical isolates from bovine mastitis (10, 38) and 8 coagulase-negative non-aureus species of Staphylococcus. The MRSA strains have previously been shown by motif-dependant PCR to be distinct (M. Daly, personal communication, (6, incorporated herein by reference only)). Of the 53 strains, 39 were successfully lysed by phage K as indicated by phage spot test and confirmed by plaque assay (Table 1). Plaque sized generally ranged from 1-1.5 mm in diameter. 14 of the strains from the MRSA group were relatively insensitive to phage K in the initial challenge (Table 1). Plaque formation did not occur with any of these using phage K although, there was inhibition in the lawn of bacterial growth, typically at phage concentrations of 108, 107 and 108 p.f.u./ml by using the plaque assay technique. This inhibition of growth in the lower dilutions of phage K plaque occurred with all the apparently insensitive MRSA strains. When phage K was incubated with these strains in broth, modified phage K variants, which were capable of forming clear plaques on their respective hosts could be obtained for all of the 14 insensitive strains (Table 1). This essentially indicated that restriction/modification (a phage resistance system (27)) is the principal cause of the phage insensitivity in the 14 isolates (48). A more effective approach to killing phage resistant staphylococcal strains is to clone and over-express the lysin enzyme from the genome of phage K.

LysK inhibits MRSA strain DPC5645 in Zymographic analysis.

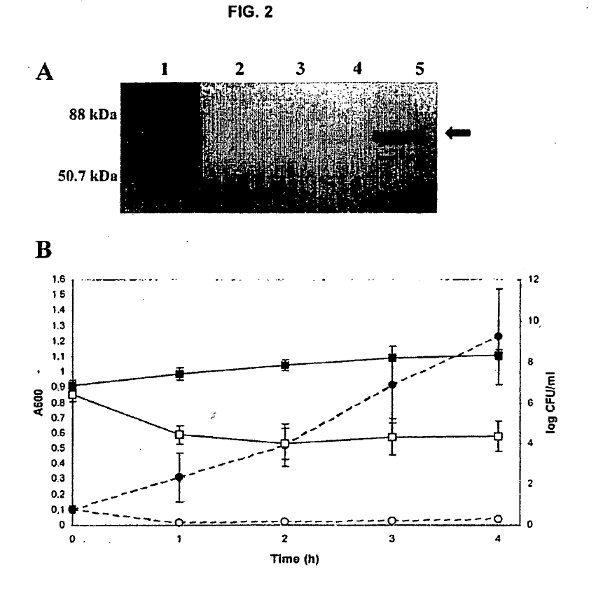

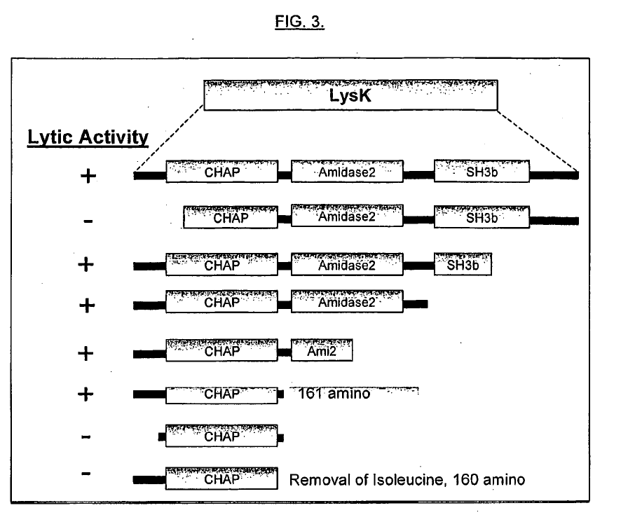

To investigate lysin activity and expression, zymographic analysis was performed as described previously (50, incorporated herein by reference only) with heat-killed strain DPC5645 (a MRSA strain isolated from an Irish hospital) embedded in the resolving gel. Mid-log(A600, 0.5) phase cells of L. lactis NZ9800-pSOFLysK and the control L. lactis NZ9800-pNZ8048 were induced for 4 h with 50 ng of nisin/ml of culture after which 1.5 ml samples were collected. Following sonication the samples were subjected to zymogramic analysis on PAGE gels containing autoclaved DPC5645 cells. Upon renaturing, a lytic zone of clearing was evident at 54 kDa in the lane containing pSOFLysK induced with nisin (FIG. 2A, lane 5), corresponding to the predicted molecular mass of LysK, unlike the uninduced control where the zone was much fainter (FIG. 2A, lane 4) and no lytic zones were evident in the lanes containing the vector control (FIG. 2A, lanes 2 and 3). These results confirmed that recombinant LysK from lactococci is enzymatically active and capable of degrading staphylococcal cell walls.

### Lactococcal Lysates Containing Lysk Kill a Wide Range of Staphylococci.

To obtain lactococcal lysates containing staphylococcal LysK, mid-log (A600, 0.5) phase cells of L. lactis NZ9800-pSOFLysK and the control L. lactis NZ9800-pNZ8048 were induced for 4 h with 50 ng of nisin/ml of culture. Cells were washed twice in sterile distilled water (SDW) and the final pellet from a 200 ml culture was then resuspended in 5 ml of SDW. 1 ml volumes of cells were ribolysed 3 times for 45 sec (setting 4.5 with 2 min intervals on ice, Hybaid, Middlesex, UK) to obtain crude lysate. Following lysis, samples were centrifuged at 10,000×g for 10 min at 4° C. and supernatants stored at −20° C.

Initially crude LysK activity was assessed for its ability to form lytic zones on autoclaved staphylococci. Bacterial strains used for host range analysis are held in the Dairy Products Research Centre culture collection and are listed in Table 2. An overnight autoclaved 50 ml culture of each staphylococcal strain (Table 2) was centrifuged and the pellet added to a 10 ml molten agar (0.7% wt/vol) overlay based on BHI medium. Samples were mixed and poured into two petri dishes to make a ‘zymogram plate’. After the overlay had solidified 10 μl aliquot of lysates were spotted onto the surface and plates scored for lytic activity. Both coagulase positive and negative staphylococci as well as drug resistant strains were inhibited by lysin containing lactococcal extract (Table 2).

Subsequently, lactococcal lysates containing LysK was assessed for their ability to form a clearing on live staphylococcal strains (Table 2). In addition, strains belonging to other genera (Table 2) were tested for sensitivity to crude LysK. Lysates from untreated L. lactis NZ9800-pSOFlysK and induced/untreated L. lactis NZ9800-pNZ8048 were used as controls. Lytic activity was scored by the intensity of the zone after overnight incubation at 37° C. In addition to lysing dead staphylococcal cells lactococcal lysates were active against a wide variety of live staphylococci including bovine mastitis strains, MRSA strains from Irish hospitals, heterogeneous-vancomycin and vancomycin resistant S. aureus and also teicoplanin resistant strains (Table. 2). A variation in lytic capabilities was evident against these staphylococcal strains. The lysin containing lactococcal extract was incapable of lysing other gram-positive bacteria such as Listeria innocua, Bacillus cereus, Lactobacillus rhamnosus and Lactobacillus paracasei.

As such this recombinant enzyme may be very useful for lysing live and dead staphylococci for diagnostic applications.

The effect of crude LysK from induced (as described above) L. lactis NZ9800-pSOFlysK was tested against an exponentially growing S. aureus strain DPC5645. Crude lysates from the induced L. lactis NZ9800-pNZ8048 were included as a negative control. S. aureus strain DPC5645 (3 mls) was grown to an OD of approximately 0.1 at 600 nm, when 500 μl of the lactococcal extract containing LysK was added. In kill curves using a human MRSA strain (DPC5645), a 99% reduction in staphylococcal cell numbers was observed 1 h after the addition of lysates containing LysK (FIG. 2B), demonstrating that recombinant LysK is capable of killing live pathogenic staphylococci.

### The Chap Domain of Lysk Retains Full Lytic Activity

Bioinformatic analysis of LysK (495 amino acids) suggests that it has a modular structure, containing two peptidoglycan hydrolase domains, CHAP (endopeptidase activity) and Amidase_{—}2 (N-acetylmuramoyl-L-alanine amidase activity), at the N-terminus and a cell-wall binding domain at the C-terminus (SH3b). Analysis of deletion derivatives of LysK confirmed that while Amidase_{—}2 and SH3b domains had no significant activity alone, the CHAP domain was as active as the intact LysK, displaying an identical lytic spectra when examined by zymographic assays. In fact, the absence of the SH3b and Amidase domains appears to result in an increase in enzyme potency. While attempting to define the smallest possible functional CHAP domain with antimicrobial activity, it was found that the endopeptidase activity associated with the CHAP domain is contained within the first 161 amino acids of LysK. Further C- or N-terminal deletions resulted in a complete loss of antimicrobial activity. The 161 amino acid truncated CHAP has been found to be active against the main MRSA strains emerging in hospitals in the local area. Site-directed mutagenesis of putative active site amino acids within this 161 amino acid truncated protein demonstrated that Cys54 is crucial for CHAP lytic activity.

### Discussion

With the increased incidence of community-acquired and hospital-acquired drug resistant staphylococci, the need for new approaches to combat this versatile pathogen is paramount. Phage K is a polyvalent or broad-host-range anti-staphylococcal phage. Based on morphology, phage K has previously been assigned to the family Myoviridae order Claudoviride (1, incorporated herein by reference only). In this study we demonstrate that phage K inhibits 9 different species of Staphylococcus, namely, S. aureus, S. epidermidis, S. saprophyticus, S. chromogenes, S. captis, S. hominis, S. haemolyticus, S. caprea and S. hyicus. Within S. aureus, it is inhibitory to a wide range of distinct strains from different hospital sources which were isolated over a three year period and also veterinary sources and hence, which we feel are representative of the problematic strains presently associated with infections in Ireland. Of particular interest is the inhibitory effect on recently emerged methicillin-resistant strains (obtained from hospital staff, out-patients and in-patients). These studies show that while phage K did not initially clearly exhibit a killing effect on all MRSA strains, it could be modified to hit the less-sensitive strains with better efficiency especially in the case of the MRSA strains simply by passing the phage through the target strain, which ordinarily would not allow plaque formation.

Elucidation of the genomic sequence of phage K lead to the identification of the gene encoding the bacterial cell-wall-degrading enzyme LysK. This gene was subsequently cloned in the expression vector pNZ8048 to give the recombinant plasmid pSOFLysK in the bacterial host Lactococcus lactis NZ9800 and thus designated Lactococcus lactis NZ9800-pSOFLysK. The LysK protein exhibited broad spectrum antibacterial activity against a wide range of staphylococci.

While a number of studies have characterised staphylococcal lysins (44, incorporated herein by reference only), to our knowledge none which have been cloned have been reported to have a broad spectrum of activity within the genus against live cells. In the present study, a genetically modified lactic acid bacteria over-expressing LysK was constructed. Expression in L. lactis yielded a protein with an apparent molecular mass of 54 kDa, which corresponds to the predicted molecular weight of LysK. Lysates containing LysK killed a wide range of staphylococci, including problematic strains such as MRSA and pathogenic S. aureus strains associated with bovine mastitis. A difference in lytic ability was observed with different staphylococcal strains, possibly reflecting differences in the cell wall composition between strains. However, other gram-positive bacteria from different genera including beneficial probiotic strains were not affected by lysates containing LysK, suggesting LysK is specific to the genus Staphylococcus. This specificity of LysK is potentially advantageous for prophylactic and/or therapeutic purposes. In conclusion, the recombinant protein retains the broad spectrum within the Staphylococcus genus of the phage itself, suggesting that it could have widespread applications as a therapeutic for infections associated with staphylococci.

TABLE 1
Lytic spectrum of Phage K sensitivity and details of bacterial strains

| | | | | | | Phage | EOP after |
|---|---|---|---|---|---|---|---|
| | | | Methicillin | Phage | | sensitivity after | phage |
| Host | Strain | Strain Details | Sensitivity | sensitivity | EOP | modification | modification |
| S. aureus | 8325 | Type strain^{a} | S | + | nc | | |
| S. aureus | St3550 | Teicoplanin | S | + | 0.087 | | |
| | | resistant^{a} | | | | | |
| S. aureus | St2573 | Teicoplanin | R | + | 0.11 | | |
| | | resistant^{a} | | | | | |
| S. aureus | Mu50 | VRSA^{a} | R | + | nc | | |
| S. aureus | Mu3 | hVRSA^{a} | R | + | nc | | |
| S. aureus | M249318 | Human MRSA^{b} | R | − | — | + | 6.75 × 10⁻³ |
| S. aureus | W64352 | Human MRSA^{b} | R | − | — | + | 2.3 × 10⁻¹ |
| S. aureus | W65216 | Human MRSA^{b} | R | − | — | + | 2.8 × 10⁻⁴ |
| S. aureus | M231003 | Human MRSA^{b} | R | + | 1.03 × 10⁻¹ | | |
| S. aureus | M249180 | Human MRSA^{b} | R | − | — | + | 1.09 × 10⁻³ |
| S. aureus | MS811 | Human MRSA^{b} | R | − | — | + | 2.26 × 10⁻³ |
| S. aureus | DPC5646 | Human MRSA^{b} | R | + | 0.77 | | |
| S. aureus | DPC5645 | Human MRSA^{b} | R | + | 0.45 | | |
| S. aureus | DPC5647 | Human MRSA^{b} | R | + | 8.46 × 10⁻ [symbol] | | |
| S. aureus | M249954 | Human MRSA^{c} | R | + | 1.12 × 10⁻¹ | | |
| S. aureus | M250594 | Human MRSA^{c} | R | + | 3.23 × 10⁻¹ | | |
| S. aureus | M254959 | Human MRSA^{c} | R | − | — | + | 7.33 × 10⁻⁵ |
| S. aureus | M255039 | Human MRSA^{c} | R | − | — | + | 1 |
| S. aureus | M255409 | Human MRSA^{c} | R | − | — | + | 7.6 × 10⁻² |
| S. aureus | M253472 | Human MRSA^{c} | R | + | 1 | | |
| S. aureus | M249739 | Human MRSA^{c} | R | + | 1.57 × 10⁻¹ | | |
| S. aureus | M249892 | Human MRSA^{c} | R | + | 4.10 × 10⁻¹ | | |
| S. aureus | M252776 | Human MRSA^{c} | R | + | 1 | | |
| S. aureus | M251760 | Human MRSA^{c} | R | + | 1.32 × 10⁻¹ | | |
| S. aureus | W71683 | Human MRSA^{c} | R | + | 5.89 × 10⁻² | | |
| S. aureus | M253206 | Human MRSA^{c} | R | + | 8.57 × 10⁻² | | |
| S. aureus | W73365 | Human MRSA^{c} | R | − | — | + | 3.4 × 10⁻¹ |
| S. aureus | M253470 | Human MRSA^{c} | R | + | 6.93 × 10⁻¹ | | |
| S. aureus | M249025 | Human MRSA^{c} | R | + | 1.41 × 10⁻¹ | | |
| S. aureus | M249138 | Human MRSA^{c} | R | + | 1 | | |
| S. aureus | M249807 | Human MRSA^{c} | R | + | 1.48 × 10⁻¹ | | |
| S. aureus | M250108 | Human MRSA^{c} | R | + | 7.3 × 10⁻¹ | | |
| S. aureus | M249671 | Human MRSA^{c} | R | − | — | + | 1.46 × 10⁻¹ |
| S. aureus | W69939 | Human MRSA^{c} | R | − | — | + | 1.177 × 10⁻³ |
| S. aureus | M253164 | Human MRSA^{c} | R | − | — | + | 2.65 × 10⁻² |
| S. aureus | M249678 | Human MRSA^{c} | R | − | — | + | 1.75 × 10⁻¹ |
| S. aureus | M251955 | Human MRSA^{c} | R | − | — | + | 2.1 × 10⁻¹ |
| S. aureus | M250564 | Human MRSA^{c} | R | + | 5.1 × 10⁻³ | | |
| S. aureus | MM77438 | Human MRSA^{c} | R | + | 2.76 × 10⁻³ | | |
| S. aureus | MM257671 | Human MRSA^{c} | R | + | 5.46 × 10⁻³ | | |
| S. aureus | MM234150 | Human MRSA^{c} | R | + | 6.2 × 10⁻¹ | | |
| S. aureus | DPC5245 | Bovine^{d} | S | + | 1 | | |
| S. aureus | DPC5246 | Bovine^{d} | S | + | 1 | | |
| S. aureus | DPC5247 | Bovine^{d} | S | + | 1 | | |
| S. aureus | DPC5971 | Bovine^{d} | S | + | 0.21 | | |
| S. epidermidis | DPC6010^{a} | Bovine^{d} | S | + | 0.46 | | |
| S. saprophyticus | DPC6011^{a} | Bovine^{d} | S | + | 0.025 | | |
| S. chromogenes | DPC6012^{a} | Bovine^{d} | S | + | 0.16 | | |
| S. captis | DPC6013^{a} | Bovine^{d} | S | + | nc | | |
| S. hominis | DPC6014^{a} | Bovine^{d} | S | + | 2.1 × 10⁻³ | | |
| S. haemolyticus | DPC6015^{a} | Bovine^{d} | S | + | nc | | |
| S. caprea | DPC6016^{a} | Bovine^{d} | S | + | 0.022 | | |
| S. hyicus | DPC6017^{a} | Bovine^{d} | S | + | 0.087 | | |

[symbol]

Coagulase-negative. + Phage sensitive as evidenced by spot assay, − Not phage sensitive as evidenced by spot assay, EOP—efficiency of plating, nc—not countable (plaques too small to count but confluent lysis, at >10⁷ p.f.u/ml), S—Sensitive to 5 μg/ml methicillin, R—Resistant to 5 μg/ml methicillin. [symbol]
indicates data missing or illegible when filed

TABLE 2
Lytic spectrum of LysK

| | | Result for cells: | | | |
|---|---|---|---|---|---|
| | | Autoclaved^{a} | | Live^{b} | |
| | | Uninduced | Induced | Uninduccd | Induced |
| Strain | Description^{c} | (pSOFLysK) | (pSOFLysK) | (pSOFLysK) | (pSOFLysK) |
| DPC 5245 | Bovine Staphylococcus aureus | − | + | − | +++ |
| DPC 5246 | Bovine S. aureus | − | + | − | +++ |
| DPC 5247 | Bovine S. aureus | − | + | − | +++ |
| DPC 5645 | MRSA | − | + | − | + |
| DPC 5646 | MRSA | − | + | − | ++ |
| DPC 5647 | MRSA | − | + | − | ++ |
| Mu3 | hVRSA | − | + | − | ++ |
| Mu50 | VRSA | − | + | − | + |
| st2573 | Teicoplanin-resistant S. aureus | − | + | − | ++ |
| st3350 | Teicoplanin-resistant S. aureus | − | + | − | +++ |
| DPC6010 | Staphylococcus epidermidis | − | + | − | ++ |
| DPC6011 | Staphylococcus saprophyticus | − | + | − | +++ |
| DPC6012 | Staphylococcus chromogenes | − | + | − | ++ |
| DPC6013 | Staphylococcus capitis | − | + | − | + |
| DPC6014 | Staphylococcus hominis | − | + | − | ++ |
| DPC6015 | Staphylococcus haemolyticus | − | + | − | ++ |
| DPC6016 | Staphylococcus caprae | − | + | − | ++ |
| DPC6017 | Staphylococcus hyicus | − | + | − | + |
| NZ9800 | Lactococcus lactis | | | − | − |
| MG1363 | L. lactis | | | − | − |
| ATCC 53103 | Lactobacillus rhamnosus | | | − | − |
| NFBC 338 | Lactobacillus paracasei | | | − | − |
| DPC3306 | Listeria innocua | | | − | − |
| DPC6087 | Bacillus cereus | | | − | − |
| P1432 | Nontoxic Escherichia coli | | | − | − |
| | O157: H7 | | | | |
| DPC6053 | E. coli JM109 (K12) | | | − | − |
| DPC6046 | Salmonella enterica DT104 | | | − | − |

^{a}Lytic zone (+) or no lytic zone (−) on zymogram plates.
^{b}Strong (+++), medium (++), or weak (+) lytic zones as indicated by the pictures on the right. − no lytic zone.
^{c}hVRSA, heterogeneous vancomycin-resistant S. aureus. VRSA, vancomycin-resistant S. aureus.

TABLE 3

List of oligonucleotides used in the
construction of plasmids

| Oligonucleotide | Oligonucleotide sequence^{a} |
|---|---|
| name | (from 5′ end to 3′ end) |
| LysK F | GCCCATGGCTAAGACTCAAGCAG |
| LysK R | GCAGATCTTTTGAATACTCCCCAGG |
| LysK CHAP F | CATGCCATGGCTAAGACTCAAGCAG |
| LysK CHAP R1 | GGAAGATCTCTATATTTCAATGAAGTGAGT |
| LysK CHAP R2 | GGAAGATCTCTATTCAATGAAGTGAGTTAAT |
| LysK Amid_2 F | CATGCCATGGCGGTATTTACATCCGGTAG |
| LysK Amid_2 R | GGAAGATCTCTAACCTATCCAAATGTGACC |
| LysK SH3b F | CATGCCATGGAATTTGTACCAACTGC |
| LysK SH3b R | GGAAGATCTCTATTTGAATACTCCCCAGGC |
| N-t R | GCTATCTACTGTTCCTTT |
| CHAP_Ra | GCTATCTACTGGTCCTTT^{b} |
| CHAP F soe | AGGAACAGTAGATAGCGAAGCAGGAGCCATT^{b} |

^{a}NcoI and BglII sites are underlined
^{b}Boldface represents an overhang that is the reverse complement of the corresponding primer for SOEing PCR

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

The words “comprises/comprising” and the words “having/including” when used herein with reference to the present invention are used to specify the presence of stated features, integers, steps or components but does not preclude the presence or addition of one or more other features, integers, steps, components or groups thereof.

### REFERENCES

1. Ackermann, H. W., and M. S. DuBow. 1987. Viruses of Prokaryotes., vol. 1. CRC Press.
2. Alisky, J., K. Iczkowski, A. Rapoport, and N. Troitsky. 1998. Bacteriophages show promise as antimicrobial agents. J Infect 36:5-15.
3. Breithaupt, H. 1999. The new antibiotics. Nat Biotechnol 17:1165-9.
4. Burnet, F. M., and D. Lush. 1935. The staphylococcal bacteriophages. J. Path. Bact. 40:455-469.
5. Carlton, R. M. 1999. Phage therapy: past history and future prospects. Arch Immunol Ther Exp (Warsz) 47:267-74.
6. Cotter, L., M. Daly, P. Greer, B. Cryan, and S. Fanning. 1998. Motif-dependent DNA analysis of a methicillin-resistant Staphylococcus aureus collection. Br J Biomed Sci 55:99-106.
7. Deshpande, L. M., T. R. Fritsche, and R. N. Jones. 2004. Molecular epidemiology of selected multidrug-resistant bacteria: a global report from the SENTRY Antimicrobial Surveillance Program. Diagn Microbiol Infect Dis 49:231-6.
8. Duckworth, D. H., and P. A. Gulig. 2002. Bacteriophages: potential treatment for bacterial infections. BioDrugs 16:57-62.
9. Fischetti, V. A. 2001. Phage antibacterials make a comeback. Nat Biotechnol 19:734-5.
10. Fitzgerald, J. R., W. J. Meaney, P. J. Hartigan, C. J. Smyth, and V. Kapur. 1997. Fine-structure molecular epidemiological analysis of Staphylococcus aureus recovered from cows. Epidemiol Infect 119:261-9.
11. Gratia, A., and M. De Namur. 1922. Individualite des principes lytiques staphylococciques de provenances differentes. Comptes Rendus de la Societe de Biologie 87:364-366.
12. Harley, J. P. 2004. The effects of chemical agents on bacteria II: Antimicrobial agents (Kirby-Bauer method). McGraw-Hill.
13. Hiramatsu, K. 2001. Vancomycin-resistant Staphylococcus aureus: a new model of antibiotic resistance. Lancet Infect Dis 1:147-55.
14. Hiramatsu, K., L. Cui, M. Kuroda, and T. Ito. 2001. The emergence and evolution of methicillin-resistant Staphylococcus aureus. Trends Microbiol 9:486-93.
15. Hotchin, J. E. 1954. The purification and electron microscopical examination of the structure of staphylococcal bacteriophage K. J Gen Microbiol 10:250-260.
16. Hotchin, J. E. 1951. Staphylococcus aureus and Staphylococcus K phage. J. Gen. Microbiol. 5:609-618.
17. Hotchin, J. E., I. M. Dawson, and W. J. Elford. 1952. The use of empty bacterial membranes in the study of the adsorption of Staphylococcus K phage upon its host. British Journal of Experimental pathology 33:177-182.
18. Kreuger, A. P., and J. H. Northrop. 1930-1. The Kinetics of the Bacterium-bacteriophage reaction. J. Gen. Physiol. 14:223.
19. Krylov, V. N. 2001. Phage Therapy in terms of bacteriophage genetics: Hopes, prospects, safety, limitations. Russian journal of genetics 37:715-730.
20. Lowy, F. D. 2003. Antimicrobial resistance: the example of Staphylococcus aureus. J. Clin Invest 111:1265-73.
21. Matsuzaki, S., M. Yasuda, H. Nishikawa, M. Kuroda, T. Ujihara, T. Shuin, Y. Shen, Z. Jin, S. Fujimoto, M. D. Nasimuzzaman, H. Wakiguchi, S. Sugihara, T. Sugiura, S. Koda, A. Muraoka, and S. Imai. 2003. Experimental protection of mice against lethal Staphylococcus aureus infection by novel bacteriophage phi MR111. J Infect Dis 187:613-24.
22. Merril, C. R., D. Scholl, and S. L. Adhya. 2003. The prospect for bacteriophage therapy in Western medicine. Nat Rev Drug Discov 2:489-97.
23. Naylor, A. R., P. D. Hayes, and S. Darke. 2001. A prospective audit of complex wound and graft infections in Great Britain and Ireland: the emergence of MRSA. Eur J Vasc Endovasc Surg 21:289-94.
24. Noble, W. C. 1998. Staphylococcal Diseases, p. 231-256. In L. Collier, A. Balows, and M. Sussman (ed.), Topley and Wilsons Microbiology and Microbial Infections, 9 ed, vol. 3.
25. O'Flaherty, S., A. Coffey, R. Edwards, W. Meaney, G. F. Fitzgerald, and R. P. Ross. 2004. Genome of Staphylococcal Phage K: a New Lineage of Myoviridae Infecting Gram-Positive Bacteria with a Low G+C Content. J Bacteriol 186:2862-2871.
26. O'Flynn, G., R. P. Ross, G. F. Fitzgerald, and A. Coffey. 2004. Evaluation of a cocktail of three bacteriophages for biocontrol of Escherichia coli O157:H7. Appl. Environ. Microbiol. 70:3417-24.
27. O'Sullivan, D., D. P. Twomey, A. Coffey, C. Hill, G. F. Fitzgerald, and R. P. Ross. 2000. Novel type I restriction specificities through domain shuffling of HsdS subunits in Lactococcus lactis. Mol Microbiol 36:866-75.
28. Rees, P. J., and B. A. Fry. 1981. The morphology of staphylococcal bacteriophage K and DNA metabolism in infected Staphylococcus aureus. J Gen Virol 53:293-307.
29. Rees, P. J., and B. A. Fry. 1981. The replication of bacteriophage K DNA in Staphylococcus aureus. J Gen Virol 55:41-51.
30. Rees, P. J., and B. A. Fry. 1983. Structure and properties of the rapidly sedimenting replicating complex of staphylococcal phage K DNA. J Gen Virol 64 (Pt 1):191-8.
31. Rippon, J. E. 1956. The classification of bacteriophages lysing Staphylococci. Journal of Hygiene 54:213-226.
32. Rountree, P. M. 1949. The serological differentiation of Staphylococcal bacteriophages. J Gen Microbiol 3:164-173.
33. Rubin, R. J., C. A. Harrington, A. Poon, K. Dietrich, J. A. Greene, and A. Molduddin. 1999. The economic impact of Staphylococcus aureus infection in New York City hospitals. Emerg Infect Dis 5:9-17.
34. O'Flaherty S., A. Coffey, W. Meaney, G. F. Fitzgerald, R. P Ross R P. 2005 The recombinant phage lysin LysK has a broad spectrum of lytic activity against clinically relevant staphylococci, including methicillin-resistant Staphylococcus aureus. J. Bacteriol. 187:7161-4.
35. Sharp, R. 2001. Bacteriophages: biology and history. J Chem Technol Biotechnol 76:667-672.
36. Sulakveidze, A., Z. Alavidze, and J. G. Morris. 2001. Bacteriophage Therapy. Antimicrobial Agents and Chemotherapy 45:649-659.
37. Tiemersma, E. W., S. L. A. M. Bronzwaer, O. Lyytikainen, J. E. Degener, P. Schrijnemakers, N. Bruinsma, J. Monen, W. Witte, H. Grundmann, and E. A. R. S. S. Participants. 2004. Methicillin-resistant Staphylococcus aureus in Europe, 1999-2002. Emerging Infectious Diseases 10:1627-1634.
38. Twomey, D. P., A. I. Wheelock, J. Flynn, W. J. Meaney, C. Hill, and R. P. Ross. 2000. Protection against Staphylococcus aureus mastitis in dairy cows using a bismuth-based teat seal containing the bacteriocin, lacticin 3147. J Dairy Sci 83:1981-8.
39. Nelson, D., L. Loomis, and V. A. Fischetti. 2001. Prevention and elimination of upper respiratory colonization of mice by group A streptococci by using a bacteriophage lytic enzyme. Proc. Natl. Acad. Sci. USA 98:4107-12.
40. Loeffler, J. M., and V. A. Fischetti. 2003. Synergistic lethal effect of a combination of phage lytic enzymes with different activities on penicillin sensitive and -resistant Streptococcus pneumoniae strains. Antimicrob. Agents Chemother. 47:375-7.
41. Schuch, R., D. Nelson, and V. A. Fischetti. 2002. A bacteriolytic agent that detects and kills Bacillus anthracis. Nature 418:884-9.
42. Sonstein, S. A., J. M. Hammel, and A. Bondi. 1971. Staphylococcal bacteriophage-associated lysin: a lytic agent active against Staphylococcus aureus. J. Bacteriol. 107:499-504.
43. Ralston, D. J., B. S. Baer, M. Lieberman, and A. P. Krueger. 1955. Virolysin: a virus-induced lysin from staphylococcal phage lysates. Proc. Soc. Exp. Biol. Med. 89:502-7.
44. Loessner, M. J., S. Gaeng, G. Wendlinger, S. K. Maier, and S. Scherer. 1998. The two-component lysis system of Staphylococcus aureus bacteriophage Twort: a large TTG-start holin and an associated amidase endolysin. FEMS Microbiol. Lett. 162:265-74.
45. Loessner, M. J., S. Gaeng, and S. Scherer. 1999. Evidence for a holin-like protein gene fully embedded out of frame in the endolysin gene of Staphylococcus aureus bacteriophage J. Bacteriol. 181:4452-60.
46. Navarre, W. W., H. Ton-That, K. F. Faull, and O, Schneewind. 1999. Multiple enzymatic activities of the murein hydrolase from staphylococcal phage phi11. Identification of a D-alanyl-glycine endopeptidase activity. J. Biol. Chem. 274:15847-56.
47. Bon, J., N. Mani, and R. K. Jayaswal. 1997. Molecular analysis of lytic genes of bacteriophage 80 alpha of Staphylococcus aureus. Can. J. Microbiol. 43:612-6.
48. O'Flaherty, S., R. P. Ross, W. Meaney, G. F. Fitzgerald, M. F. Elbreki, and A. Coffey. 2005. Potential of the polyvalent anti-Staphylococcus bacteriophage K for the control of antibiotic-resistant staphylococci from hospitals. Appl. Environ. Microbiol. 71, 1836-1842.
49. de Ruyter, P. G., O. P. Kuipers, and W. M. de Vos. 1996. Controlled gene expression systems for Lactococcus lactis with the food-grade inducer nisin. Appl. Environ. Microbiol. 62:3662-7.
50. Hickey, R. M., D. P. Twomey, R. P. Ross, and C. Hill. 2003. Production of enterolysin A by a raw milk enterococcal isolate exhibiting multiple virulence factors. Microbiology 149:655-64.
51. Yoong, P., R. Schuch, D. Nelson and V. A. Fischetti. 2004. Identification of a broadly active phage lytic enzyme with lethal activity against antibiotic-resistant Enterococcus faecalis and Enterococcus faecium. J. Bacteriol. 186: 4808-12.

1. A plasmid providing anti-staphylococcal lysin activity as deposited with NCIMB under accession No. NCIMB 41409 and plasmids substantially similar thereto also providing anti-staphylococcal lysin activity.

2. A gene encoding anti-staphylococcal lysin activity as deposited in plasmid pSOFLysK with the NCIMB under accession NCIMB 41409 of claim 1, genes substantially similar thereto encoding anti-staphylococcal activity and truncated derivatives thereto encoding anti-staphylococcal activity.

3. An anti-staphylococcal lysin encoded by a plasmid providing anti-staphylococcal lysin activity as deposited with NCIMB under accession No. NCIMB 41409 and plasmids substantially similar thereto also providing anti-staphylococcal lysin activity, or a gene encoding anti-staphylococcal lysin activity as deposited in plasmid pSOFLysK with the NCIMB under accession NCIMB 41409, genes substantially similar thereto encoding anti-staphylococcal activity and truncated derivatives thereto encoding anti-staphylococcal activity.

4. A peptide comprising the N-terminal about 161 amino-acid sequence of the lysin as claimed in claim 3.

5. A peptide comprising the amino acid sequence SEQ ID No. 16.

6. A nucleotide sequence comprising a sequence encoding the peptide as claimed in claim 4 or claim 5.

7. A chimeric protein molecule comprising the N-terminal about 161 amino-acid sequence as claimed in claim 4.

8. (canceled)

9. A method of lysing staphylococci for diagnostic applications comprising contacting the staphylococci with
a plasmid providing anti-staphylococcal lysin activity as deposited with NCIMB under accession No. NCIMB 41409 and plasmids substantially similar thereto also providing anti-staphylococcal lysin activity;
a gene encoding anti-staphylococcal lysin activity as deposited in plasmid pSOFLysK with the NCIMB under accession NCIMB 41409, genes substantially similar thereto encoding anti-staphylococcal activity, a truncated derivative thereto encoding anti-staphylococcal activity; or
an anti-microbial lysin encoded by the plasmid, the plasmids substantially similar thereto, the gene, the genes substantially similar thereto or the truncated derivative thereto.

10. A composition comprising a plasmid providing anti-staphylococcal lysin activity as deposited with NCIMB under accession No. NCIMB 41409 and plasmids substantially similar thereto also providing anti-staphylococcal lysin activity;
a gene encoding anti-staphylococcal lysin activity as deposited in plasmid pSOFLysK with the NCIMB under accession NCIMB 41409, genes substantially similar thereto encoding anti-staphylococcal activity a truncated derivative thereto encoding anti-staphylococcal activity; or
an anti-microbial lysin encoded by the plasmid, the plasmids substantially similar thereto, the gene, the genes substantially similar thereto or the truncated derivative thereto.

11. The composition as claimed in claim 10 wherein the composition is a topical preparation selected from a hand or skin wash, a shampoo, a topical cream or a disinfecting preparation.

12. A pharmaceutical composition comprising the composition of claim 10.

13. The method of claim 9 wherein the staphylococci is selected from the group comprising: S. aureus, S. epidermidis. S. saprophytics, S. chromogenes, S. captis, S. hominis, S. haemolyticus, S. caprea S. hyicus and antibiotic resistant variants thereof and combinations thereof.

14. A method of treating topical infections in a subject comprising administering the composition of claim 10 to the subject.

15. A method of disinfecting an environment comprising delivering the composition of claim 10 to the environment.

16. A method of treating a staphylococcal infection comprising administering to a patient a pharmaceutically effective amount of a pharmaceutical composition as claimed in claim 12.

17. A vector comprising the nucleotide sequence as claimed in claim 6.

18. A host cell comprising the vector as claimed in claim 17.

19. The method of claim 16 wherein the staphylococcal infection is from a staphylococci selected from the group comprising: S. aureus, S. epidermidis, S. saprophytics, S. chromogenes, S. captis., S. hominis, S. haemolyticus, S. caprea S. hyicus and antibiotic resistant variants thereof and combinations thereof.


SEQUENCE LISTING (16 sequences)

SEQ ID NO: 1 - DNA, 54 bp, Artificial Sequence
  lysinF, forward primer used to isolate the lysK gene from phage K cDNA
cggcatgcaggaggaaaaaaaaaatggctaagactcaagcagaaataaataaac

SEQ ID NO: 2 - DNA, 29 bp, Artificial Sequence
  lysinR, reverse primer used to isolate the lysK gene from phage K cDNA
gctctagactatttgaatactccccaggc

SEQ ID NO: 3 - DNA, 23 bp, Artificial Sequence
  lysK F, forward primer used to isolate the lysK gene
gcccatggctaagactcaagcag

SEQ ID NO: 4 - DNA, 25 bp, Artificial Sequence
  lysK R, reverse primer used to isolate the lysK gene
gcagatcttttgaatactccccagg

SEQ ID NO: 5 - DNA, 25 bp, Artificial Sequence
  lysK CHAP F, forward primer used to isolate a domain of the lysK gene
catgccatggctaagactcaagcag

SEQ ID NO: 6 - DNA, 30 bp, Artificial Sequence
  lysK CHAP R1, reverse primer used to isolate a domain of the lysK gene
ggaagatctctatatttcaatgaagtgagt

SEQ ID NO: 7 - DNA, 31 bp, Artificial Sequence
  lysK CHAP R2, reverse primer used to isolate a domain of the lysK gene
ggaagatctctattcaatgaagtgagttaat

SEQ ID NO: 8 - DNA, 29 bp, Artificial Sequence
  lysK Amid_2 F, forward primer used to isolate a domain of the lysK gene
catgccatggcggtatttacatccggtag

SEQ ID NO: 9 - DNA, 30 bp, Artificial Sequence
  LysK Amid_2 R, reverse primer used to isolate a domain of the lysK gene
ggaagatctctaacctatccaaatgtgacc

SEQ ID NO: 10 - DNA, 26 bp, Artificial Sequence
  LysK  SH3b F, forward primer used to isolate a domain of the lysK gene
catgccatggaatttgtaccaactgc

SEQ ID NO: 11 - DNA, 30 bp, Artificial Sequence
  LysK  SH3b R, reverse primer used to isolate a domain of the lysK gene
ggaagatctctatttgaatactccccaggc

SEQ ID NO: 12 - DNA, 18 bp, Artificial Sequence
  N-t R, reverse primer used to isolate a domain of the lysK gene
gctatctactgttccttt

SEQ ID NO: 13 - DNA, 18 bp, Artificial Sequence
  CHAP_Ra, reverse primer used to isolate a domain of the lysK gene
gctatctactggtccttt

SEQ ID NO: 14 - DNA, 31 bp, Artificial Sequence
  CHAP F soe, forward primer used to isolate a domain of the lysK gene
aggaacagtagatagcgaagcaggagccatt

SEQ ID NO: 15 - protein, 495 aa, Unknown
  Staphylococcus phage K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 16 - protein, 100 aa, Unknown
  Staphylococcus phage K CHAP Domain
GAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYA